(19) United States
(12) Patent Application Publication
(10) Pub. No.: US 2012/0095415 A1
(43) Pub. Date: Apr. 19, 2012

(54) DRUG PORT VERIFICATION VALVE

(75) Inventors: Pierre Sharvit, Emeq Hefer (IL); Jacob Nusbacher, Ra'anana (IL); Gershon Goldenberg, Pardes Hana Carcus (IL)

(73) Assignee: PRO-IV LTD., Kfar Vitkin (IL)

(21) Appl. No.: 12/812,602

(22) PCT Filed: Dec. 17, 2008
(86) PCT No.: PCT/IL08/01616

§ 371 Date: Nov. 24, 2011

Related U.S. Application Data
(60) Provisional application No. 61/006,578, filed on Jan. 22, 2008.

(51) Int. Cl. A61M 5/00

(52) U.S. Cl. 604/244

(57) ABSTRACT

A drug port smart valve and a method of its use, for preventing medical errors when injecting IV fluids and medication into humans and animals, and, in particular to ensure authentication of medication infused in IV bags and syringes.

### FIELD OF THE INVENTION

The present invention relates to means and a method designated to prevent medical errors when injecting IV fluids and medications into humans and animals, and, in particular to ensure authentication of medications infused in IV bags and syringes.

### BACKGROUND OF THE INVENTION

An apparatus, system and method for administration of a substance is described in the International Application PCT/IL/2005/001118 of Sharvit et al., International Publication Number WO 2006/046242, which is incorporated by reference for all purposes as if fully set forth herein.

WO 2006/046242 discloses an infusion control valve adapted to be actuated by a valve actuator, an infusion valve actuator adapted to actuate an infusion control valve upon being triggered by an authentication unit and a method for the administration of a substance.

The method according to WO 2006/046242 also uses a hand-held (HHD) computer and a smart (electronic) key.

### SUMMARY OF THE INVENTION

The present invention relates to means and a method of prevention of error and ensuring authentication of medications infused in IV bags and syringes, and other authentication, such as the verification of movement of fluids in all directions from bags to vials, bags to syringes, and syringes to vials.

These means and method are according to the present invention, some of whose inventors are also inventors of WO 2006/046242, and are designated to add further security to the prior invention.

While the prior invention was designated to prevent error in the injection of medications, the present invention is designated to prevent errant infusion of medications into containers to be used for injection, particularly IV bags and syringes.

The computer and the smart key described in patent application PCT/IL2005/001118, also serve for the activation of the drug port smart valve, according to the present invention. Activation of the drug port smart valve for the passage of fluid causes computer feedback which prevents additional repetition of the same process, ensuring that no material flows mistakenly into the IV bag, vial, or syringe. In the case that the IV fluid is to be infused with medication from two or more sources, a series of injection, one after the other, should be performed, with each injection using a new drug port smart valve.

The flow through the flexible tubule of the drug port smart valve is according to the known laws of fluid flow and can be in either direction, also in the form of flow of fluid from the vial to the IV bag, and of air and fluid from the IV bag to the vial, so that it is possible for the IV fluid to dilute medicinal fluid in the vial. The flow of fluid is usually in the direction of gravitational force, other than the case of pressure being applied to the IV bag, in a direction according to the orientation of the IV bag and the vial relative to each other. The air would naturally flow normally from the high pressure area to the low pressure area.

The means and method according to the present invention can prevent errors and ensure meeting standards, such as the required standards specified in the U.S. Food and Drug Administration, Docket No. 2002N-0204, “Bar Code Label Requirement for Human Drug Products and Biological Products”, which is incorporated by reference for all purposes as if fully set forth herein.

According to some embodiments of the present invention there is provided a drug port valve for ensuring authentication of medications, in fluid state, infused in IV bags and syringes from a drug vial, under control of an authentication unit, the authentication unit containing characteristics of the medication fluid and details of the patient, for calculating a correlation value between the details and the characteristics and having a key for opening passage for the medication fluid flow through the drug port valve, the drug port valve including: (a) an immoveable assembly including: (i) a breakable lock; (ii) a flexible tube having a first end and a second end, wherein the first end of the flexible tube is disposed on the immoveable assembly; and (iii) an outlet connector disposed at the second end of the flexible tube, wherein the breakable lock can be broken by a key pin, locking the immoveable assembly; and (b) a moveable assembly having limited linear, one dimensional, motion capability, with relation to the immoveable assembly, the immoveable assembly including: (i) a shutter.

According to still further features in the described embodiments the immoveable assembly further includes: (ii) an inlet adaptor having external end, shape, and dimensions enabling the connection of a drug vial.

According to still further features in the described embodiments the moveable assembly further includes: (ii) a first integral needle having a sharp tip for enabling puncturing the drug vial's seal for enabling flow of fluid from the drug vial into the flexible tube, as a result of movement of the moveable assembly, wherein the immoveable assembly defines a three-dimensional XYZ Cartesian coordinate system, having an origin located at a geometrical center of the external end of the inlet adaptor, wherein an X axis is directed from the origin into the drug port valve, and goes through the sharp tip of the first integral needle, wherein a Z axis is perpendicular to the X axis and is on a symmetry plane with the shutter, and wherein a Y axis is perpendicular to both prior axes.

According to still further features in the described embodiments the drug port valve is configured so as to cause the shutter to press the flexible tube and to create blockage of fluid flow through the flexible tube as a result of movement of the moveable assembly in the direction −x with regard to the immoveable assembly.

According to still further features in the described embodiments the drug port valve is further configured such that breaking of the breakable lock causes the shutter to cease pressing the flexible tube and open the flexible tube for flow of fluid.

According to still further features in the described embodiments the outlet connector is configured so as to enable connection to an IV bag.

According to another embodiment the outlet connector is configured so as to enable connection to a syringe without a needle.

According to another embodiment the outlet connector is configured so as to enable connection to a syringe with a needle.

According to some embodiments of the present invention there is provided a method for ensuring authentication of medications, in fluid state, infused in an IV bag from a drug vial, the method including the stages of: (a) providing a drug port valve, the drug port valve including: (i) an immoveable assembly including: (A) a breakable lock; (B) a flexible tube having first end and second end, wherein the first end of the flexible tube is disposed on the immoveable assembly; and (C) an outlet connector disposed on the second end of the flexible tube, wherein the breakable lock can be broken by a key pin, locking the immoveable assembly; and (ii) a moveable assembly having limited linear, one dimensional, motion capability, with relation to the immoveable assembly, the immoveable assembly including: (A) a shutter; (b) connecting a drug vial, having a vial barcode sticker having a drug vial barcode sticker code, to the drug port valve; (c) pressing on and closing the flexible tube; (d) scanning the vial barcode sticker by a HHD computer; (e) connecting an IV bag to the drug port valve; (f) using the HHD computer for releasing the pressure and opening the flexible tube to fluid flow; and (g) printing a second barcode sticker with a code identical to the drug vial barcode sticker code, and sticking the second barcode sticker on the IV bag.

Additional objects and advantages of the invention will be set forth in part in the description which follows and, in part, will be obvious from the description, or may be learned by practice of the invention.

### BRIEF DESCRIPTION OF THE DRAWINGS

The invention is herein described, by way of example only, with reference to the accompanying drawings, wherein:

FIG. 1 is a schematic side view illustration of a first exemplary embodiment of a drug port smart valve, according to the present invention.

FIG. 2 is an exploded schematic illustration of the first exemplary embodiment of the drug port smart valve, according to the present invention.

FIG. 3a is a schematic cross sectional view of the first exemplary embodiment of the drug port smart valve, according to the present invention.

FIG. 3b is a schematic side view of the first exemplary embodiment of the drug port smart valve, according to the present invention.

FIG. 3c is a schematic perspective view of the first exemplary embodiment of the drug port smart valve, according to the present invention.

FIG. 4a is a schematic side view of the first exemplary embodiment of the drug port smart valve, according to the present invention and of a drug vial 9, which shows the first stage of the action of the drug port smart valve

FIG. 4b is a schematic perspective view of the first exemplary embodiment of the drug port smart valve, according to the present invention and of a drug vial showing the first stage of the function of the drug port smart valve.

FIG. 5 is a schematic perspective view of the first exemplary embodiment of the drug port smart valve, according to the present invention, connected to drug vial, as well as HHD computer.

FIG. 6 is a schematic side view of the first exemplary embodiment of the drug port smart valve, according to the present invention, and of the HHD computer, an IV bag, a syringe, and a syringe with a syringe needle.

FIG. 7 is a schematic perspective view of the first exemplary embodiment of the drug port smart valve, according to the present invention, in a state in which the flow of fluid is blocked, and it is connected to a drug vial, an IV bag, and a HHD computer.

FIG. 8 is a schematic perspective view of the first exemplary embodiment of the drug port smart valve, according to the present invention, which is connected to a drug vial, and an IV bag, alongside a HHD computer, and a barcode sticker printer.

FIG. 9 is a schematic side view of the first exemplary embodiment of the drug port smart valve, according to the present invention, which is connected to a drug via, alongside an IV bag.

FIG. 10a is a schematic side view of the first exemplary embodiment of the drug port smart valve, according to the present invention, alongside a drug vial. This illustration once again shows the stages of blocking and opening the passage for fluid flow.

FIG. 10b is a schematic cross sectional view of the first exemplary embodiment of the drug port smart valve, according to the present invention.

FIG. 11a is a schematic side view of the first exemplary embodiment of the drug port smart valve according to the present invention.

FIG. 11b is a schematic cross sectional view of the first exemplary embodiment of the drug port smart valve, according to the present invention.

FIG. 11c is a schematic cross sectional view of the first exemplary embodiment of the drug port smart valve, according to the present invention.

FIG. 12a is a schematic side view of the first exemplary embodiment of the drug port smart valve, according to the present invention.

FIG. 12b is a schematic cross sectional view of the first exemplary embodiment of the drug port smart valve, according to the present invention.

FIG. 13a is a schematic side view of the first exemplary embodiment of the drug port smart valve, according to the present invention.

FIG. 13b is a schematic cross sectional view of the first exemplary embodiment of the drug port smart valve, according to the present invention.

FIG. 14 is a schematic perspective view of the first exemplary embodiment of the drug port smart valve, according to the present invention, and of a HHD computer.

FIG. 15a is a schematic side view of the first exemplary embodiment of the drug port smart valve, according to the present invention, connected to drug vial, and HHD computer.

FIG. 15b is a schematic side view of details of the first exemplary embodiment of the drug port smart valve, according to the present invention.

FIG. 15c is a schematic cross sectional view of the first exemplary embodiment of the drug port smart valve, according to the present invention.

FIG. 16a is a schematic side view of the first exemplary embodiment of the drug port smart valve, connected to drug vial and HHD computer.

FIG. 16b is a schematic side view of details of the first exemplary embodiment of the drug port smart valve, according to the present invention.

FIG. 17a is a schematic side view of the first exemplary embodiment of the drug port smart valve, according to the present invention, connected to drug vial.

FIG. 17b is a schematic cross sectional view of the first exemplary embodiment of the drug port smart valve.

FIG. 17c is a schematic cross sectional view of the exemplary embodiment of the drug port smart valve, showing flow of fluid.

FIG. 18a is a schematic side view of the first exemplary embodiment of the drug port smart valve, according to the present invention, which is connected to a drug vial and an IV bag.

FIG. 18b is a schematic cross sectional view of the first exemplary embodiment of the drug port smart valve, according to the present invention, which is connected to a drug vial and an IV bag.

FIG. 18c is a schematic cross sectional view of details of the first exemplary embodiment of the drug port smart valve according to the present invention.

FIG. 19a is a schematic side view of a second exemplary embodiment of the drug port smart valve, according to the present invention, which is connected to a drug vial and a syringe without a needle.

FIG. 19b is a schematic cross sectional view of the second exemplary embodiment of the drug port smart valve, according to the present invention, which is connected to a drug vial and a syringe without a needle.

FIG. 19c is a schematic cross sectional view of details of the second exemplary embodiment of the drug port smart valve, according to the present invention.

FIG. 20a is a schematic perspective view of a third exemplary embodiment of the drug port smart valve, according to the present invention, connected to a drug vial and to a syringe equipped with a syringe needle.

FIG. 20b is a schematic cross sectional view of the third exemplary embodiment of the drug port smart valve, according to the present invention, which is connected to a drug vial and a syringe equipped with a syringe needle.

FIG. 20c is a schematic cross sectional view of details of the third exemplary embodiment of the drug port smart valve, according to the present invention.

FIG. 21a is schematic side view of the first exemplary embodiment of the drug port smart valve, according to the present invention, connected to a drug vial and to an IV bag.

FIG. 21b is a schematic cross sectional view of the first exemplary embodiment of the drug port smart valve, according to the present invention, connected to a drug vial and to an IV bag.

FIG. 22a is schematic side view of the second exemplary embodiment of the drug port smart valve, according to the present invention, connected to a drug vial and to a syringe without a needle.

FIG. 22b is a schematic cross sectional view of the second exemplary embodiment of the drug port smart valve, according to the present invention, connected to a drug vial and to a syringe without a needle.

FIG. 23a is schematic side view of the second exemplary embodiment of the drug port smart valve, according to the present invention, connected to a drug vial and to a syringe equipped with a syringe needle.

FIG. 23b is a schematic cross sectional view of the second exemplary embodiment of the drug port smart valve, according to the present invention, connected to a drug vial and to a syringe equipped with a syringe needle.

FIG. 24a is a schematic side view of both assemblies of the exemplary embodiment of the drug port smart valve, the immoveable assembly, and the moveable assembly, according to the present invention, when they are separated from each other.

FIG. 24b is a schematic cross sectional view of both assemblies of the exemplary embodiment of the drug port smart valve, the immoveable assembly, and the moveable assembly, according to the present invention, when they are separated from each other.

FIG. 24c is a schematic perspective view of both assemblies of the exemplary embodiment of the drug port smart valve, the immoveable assembly, and the moveable assembly, according to the present invention, when they are separated from each other.

FIG. 25a is a schematic side view of the exemplary embodiment of the drug port smart valve, according to the present invention, and which is connected to drug vial.

FIG. 25b is a schematic cross sectional view of the exemplary embodiment of the drug port smart valve, according to the present invention and which is connected to drug vial.

FIG. 25c is a schematic cross sectional view of the exemplary embodiment of the drug port smart valve, according to the present invention.

FIG. 26a is a schematic side view of an exemplary embodiment of the drug port smart valve, according to the present invention, connected to a drug vial, and to an “on-line” port.

FIG. 26b is a schematic side view of an exemplary embodiment of the drug port smart valve, according to the present invention, connected to a drug vial and to an IV bag. This illustration demonstrates additional options for connection of the drug port smart valve to IV bag.

FIG. 26c is a schematic cross sectional view of an exemplary embodiment of the drug port smart valve, according to the present invention, connected to a drug vial and to an IV bag.

### DETAILED DESCRIPTION OF EMBODIMENTS

The present invention is of means and a method to prevent errors and ensure authentication of medications infused in IV bags and syringes.

The principles and operation of a drug port smart valve according to the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings.

Note that with suitable adaptation, the present invention can serve to prevent errors when administering medication to humans and animal in all known forms of administration. The invention also enables prevention of error in many various methods of parenteral injection, such as: intravenous, intraarterial, intramuscular, subcutaneous, intraosseus infusion, intradermal, intrathecal intraperitoneal, and epidural.

The present invention provides a wide variety of options for transfer of liquids and/or drugs, such as: a vial with liquid drug to a drug port in an IV bag, a vial with powder to a drug port in an IV bag, a vial with liquid drug to a special drug port of an advantage system type, a vial with powder to a special drug port of an advantage system type, a vial with liquid drug to a drug port in an IV administration set line such as a port manufactured into the IV line or an added port (e.g. 3-way stopcock), a syringe without needle with liquid drug to a drug port in an IV bag, a syringe without needle with liquid drug to a port in an IV administration set line such as an integral port or an added port (e.g. 3-way stopcock), a syringe without needle with liquid (e.g. diluents) to a vial with drug (powdered or liquid), an empty syringe with needle to a vial with liquid drug, a syringe with needle with liquid drug to a drug port in an IV bag, a syringe with needle with liquid drug to a port in an IV administration set line such as an integral port or an added port (e.g. 3-way stopcock), or a syringe with needle, with diluents to a vial with drug.

The drug port smart valve can be adapted to a variety of means not designated for administering medication, as can these means be adapted to the drug port smart valve. One such means is a bottle of mother's milk. This bottle can have a barcode sticker with identification data, and can serve for giving milk pumped from a mother's breast to be given to her infant at a later time.

Unless otherwise defined, all technical and scientific terms used herein have the same meaning as commonly understood by one of ordinary skill in the art to which this invention belongs. The materials, dimensions, methods, and examples provided herein are illustrative only and are not intended to be limiting.

The following list is a legend of the numbering of the application illustrations:

1 immoveable assembly

2 moveable assembly

3 shutter

4 opening port

5 breakable lock

6 flexible (plastic) tube

7 outlet connector

7a outlet connector of first exemplary embodiment

7b outlet connector of second exemplary embodiment

7c outlet connector of third exemplary embodiment

8 “on-line” port

9 drug vial

10 vial barcode sticker

11 syringe without needle

12 syringe needle

14 diaphragm

16 first integral needle

17 IV bag

18 IV bag first port

19 IV bag second port

20 second integral needle

21 window

23 drug vial seal

24 a point on the shutter inclined plane

25 inlet port

26 shutter rear wall

27 blocking zone

28 key pin

29 lower anvil

30 a hand-held (HHD) computer

31 LCD screen

32 keypad

34 second (elastomer) seal

35 fluid free flow

37 entry opening

38 exit opening

39 shutter inclined plane

40 IR radiation

44 barcode sticker printer

46 inlet adaptor

47 outlet adaptor

49 HHD computer port

100 drug port smart valve

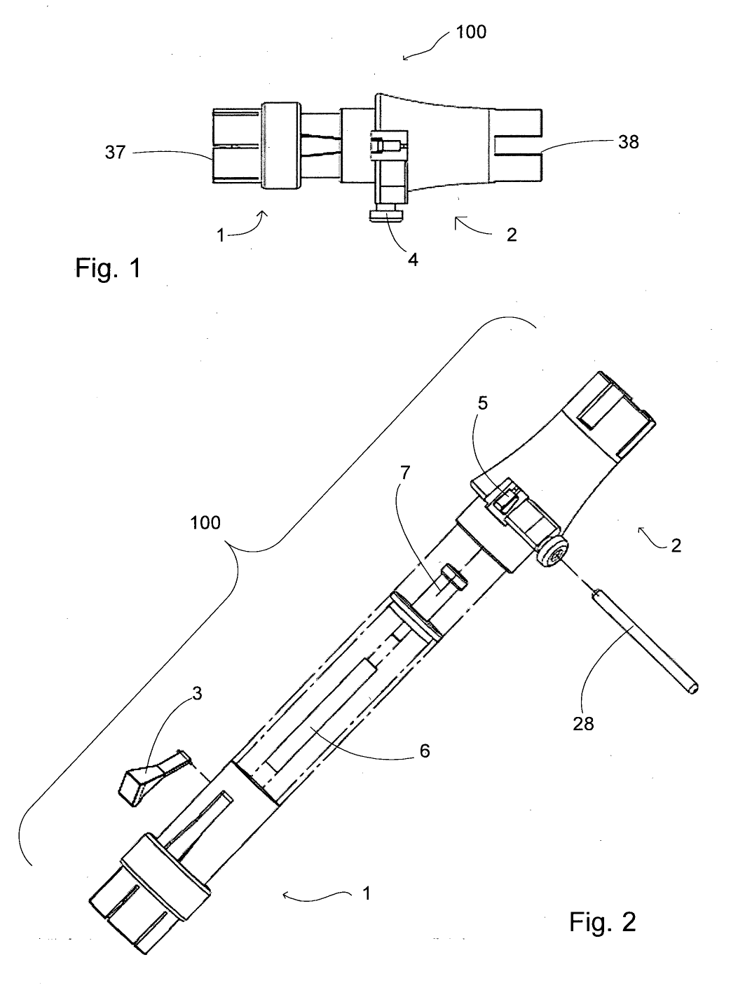

Referring now to the drawings, FIG. 1 is a schematic side view illustration of a first exemplary embodiment of a drug port smart valve 100, according to the present invention.

Drug port smart valve 100 is an interface between an IV bag and a vial, a syringe, or any other suitable container, containing medicine intended for injection into an IV bag for the purpose of infusing it in the IV fluid, which could be, for example, a medical fluid such as saline, contained within the bag, syringe, or container.

Drug port smart valve 100 is composed of two main assemblies, an immoveable assembly 1 designated to connect to the medicine container, and a moveable assembly 2 designated to connect to the IV bag, syringe, or container. The determination of immoveable assembly 1 as stationary is arbitrary and is for the purpose of easier understanding of the function of drug port smart valve 100.

The medicine flows through drug port smart valve 100. After having entered through entry opening 37, it flows through a flexible tube, not shown in this illustration, and exits through the exit opening 38.

As will be explained in the following, the motion of moveable assembly 2 upon the immoveable assembly 1 blocks the flow of medicinal fluid through the flexible tube.

Moveable assembly 2 also includes an opening port 4 serving as a connective interface to a smart key which enables flow into the flexible tube only after connecting to the smart key and examining the parameters of the medical instructions.

FIG. 2 is an exploded schematic illustration of the exemplary embodiment of the drug port smart valve 100, according to the present invention. The illustration shows flexible tube 6, which can be made of plastic material or any other suitable material, connected between the immoveable assembly 1 and outlet connector 7.

Furthermore, the following elements are shown in the illustration, shutter 3, breakable lock 5, outlet connectors 7, and key pin 28, a specific explanation of which will be presented further in the present application.

In addition, a filter, not shown in the present illustration, can be incorporated into the drug port smart valve 100.

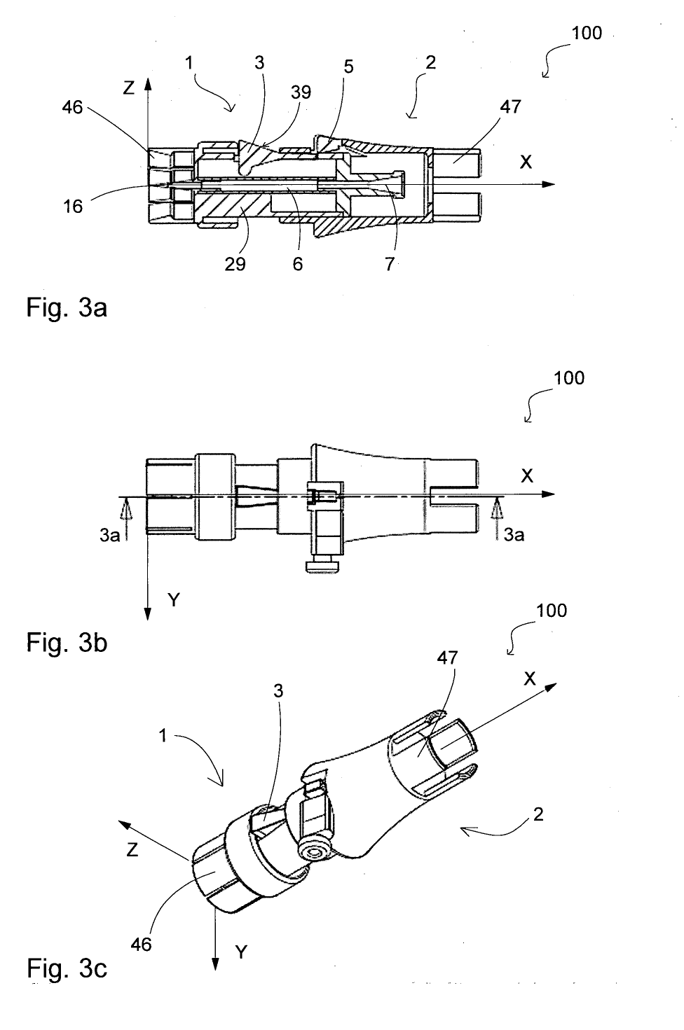

FIG. 3a is a schematic cross sectional view 3a-3a of the exemplary embodiment of the drug port smart valve 100, according to the present invention. The illustration shows a detailed description of a mechanism enabling flow and blockage of fluid.

The flexible tube 6 is connected on one end to the immoveable assembly 1 and on the other end to the flexible tube 6, which is connected to outlet connector 7, which is designated for connection to external units. There are three types of outlet connector 7, the first enables connection to an IV bag feed tube, the second enables connection to a syringe without a needle, and the third enables connection to a syringe with a needle. A breakable lock 5 is disposed upon the moveable assembly 2. The breakable lock 5 breaks when key pin 28, not shown in the illustration, is inserted into the moveable assembly 2.

When the moveable assembly 2 moves towards the immoveable assembly 1, one end, shown on the left of the illustration, of shutter 3, which is made of elastic material, moves towards the flexible tube 6, and presses it towards lower anvil 29, while the other end is harnessed to the moveable assembly 2.

After pressing the moveable assembly 2 to the immoveable assembly 1 complete blockage is achieved, preventing the flow of fluid through the flexible tube 6. Proper planning of the structure of the components of the drug port smart valve 100 creates a force balance that ensures the existence of the aforementioned blockage. Thus the required force for insertion of a medicine vial head into the inlet adaptor 46 is approximately twice the force necessary for moving the moveable assembly 2 on the immoveable assembly 1. The resistance force for this movement is achieved mainly by means of shutter 3. During this movement, a small area of the moveable assembly 2 presses upon the shutter inclined plane 39, resulting in a thrusting force perpendicular to the direction of movement, which drives the end of shutter 3 towards the flexible tube 6 and crushes it to the point of complete blockage.

Furthermore the illustration shows a first integral needle 16, which has a sharp tip.

FIG. 3b is a schematic side view of the exemplary embodiment of the drug port smart valve 100, according to the present invention. The illustration shows the plane of section 3a-3a.

FIG. 3c is a schematic perspective view of the exemplary embodiment of the drug port smart valve 100, according to the present invention. This illustration, like both previous illustrations, shows an axis system to assist in further description and particularly to assist in clear definition of the directions of motion.

The immoveable assembly 1 defines a three-dimensional XYZ Cartesian coordinate system, wherein the origin is at the geometrical center of the external end of the inlet adaptor 46. X axis is directed from the origin into the drug port smart valve 100, and goes through the sharp tip of the first integral needle 16, which is not shown in this illustration, however was described above.

X axis is the symmetry axis for those components of the drug port smart valve 100, which have rotational symmetry.

Z axis is perpendicular to X axis and is on the symmetry plane with shutter 3, while Y axis is perpendicular to both prior axes.

Each axis is perpendicular to the plane created by the other two axes, and the directions of the axes are according to the definitions of a standard right-handed axis system.

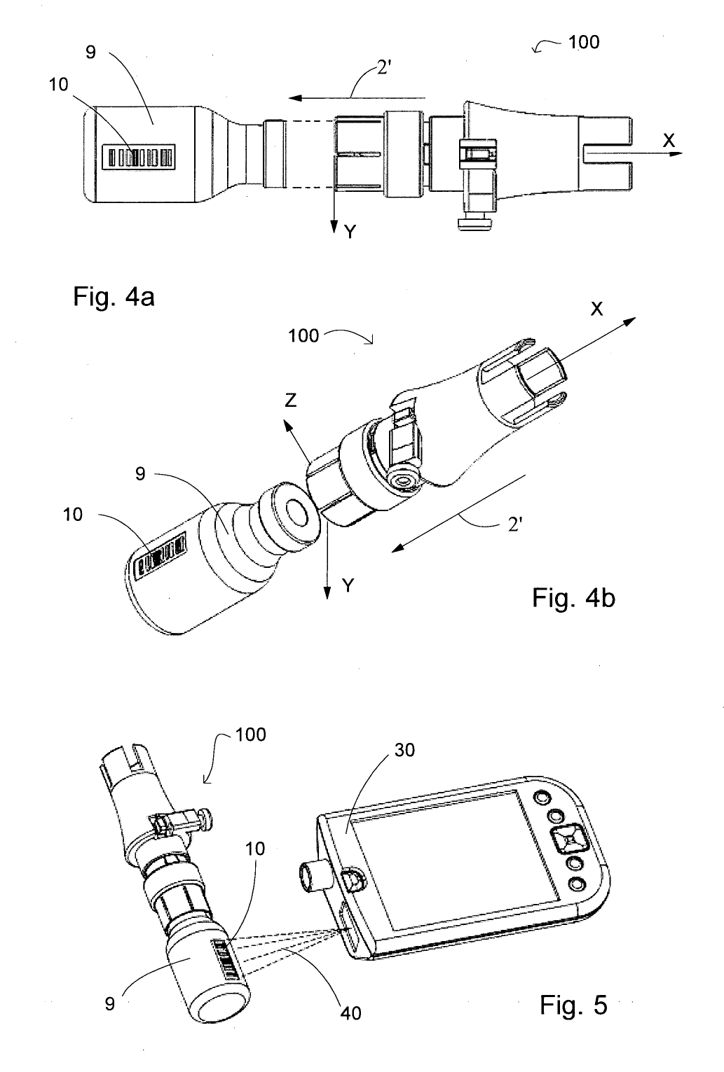

FIG. 4a is a schematic side view of the exemplary embodiment of the drug port smart valve 100, according to the present invention and of a drug vial 9, which shows the first stage of the action of the drug port smart valve 100. The first stage is the assembly of the drug port smart valve 100 to drug vial 9 with motion 2′, namely in the direction −x, with force being applied at the time of connection.

The illustration shows that vial barcode sticker 10 is attached to drug vial 9. The vial barcode sticker 10 contains information designated to identify the contents of drug vial 9 to prevent errant medication of a patient.

FIG. 4b is a schematic perspective view of the exemplary embodiment of the drug port smart valve 100, according to the present invention and of a drug vial 9 showing the first stage of the function of the drug port smart valve 100.

FIG. 5 is a schematic perspective view of the exemplary embodiment of the drug port smart valve 100, according to the present invention, connected to drug vial 9, as well as HHD computer 30. In this state, as described with regard to FIG. 3a, there is a blockage of fluid flow through the flexible tube, which is internal and is not shown in the present illustration.

As shown in the illustration, the vial barcode sticker 10 is scanned by the HHD computer 30 by means of a barcode scanner, which can also be a scanner in the IR range. The illustration shows IR radiation 40.

According to the present invention the vial can also be identified by other methods, such as, CCD camera, scanner, RFID, or any suitable barcode scanner or identifying system. To remove any doubt, the present invention is not limited to any specific type of authentification system such as barcode, RFID, or any other.

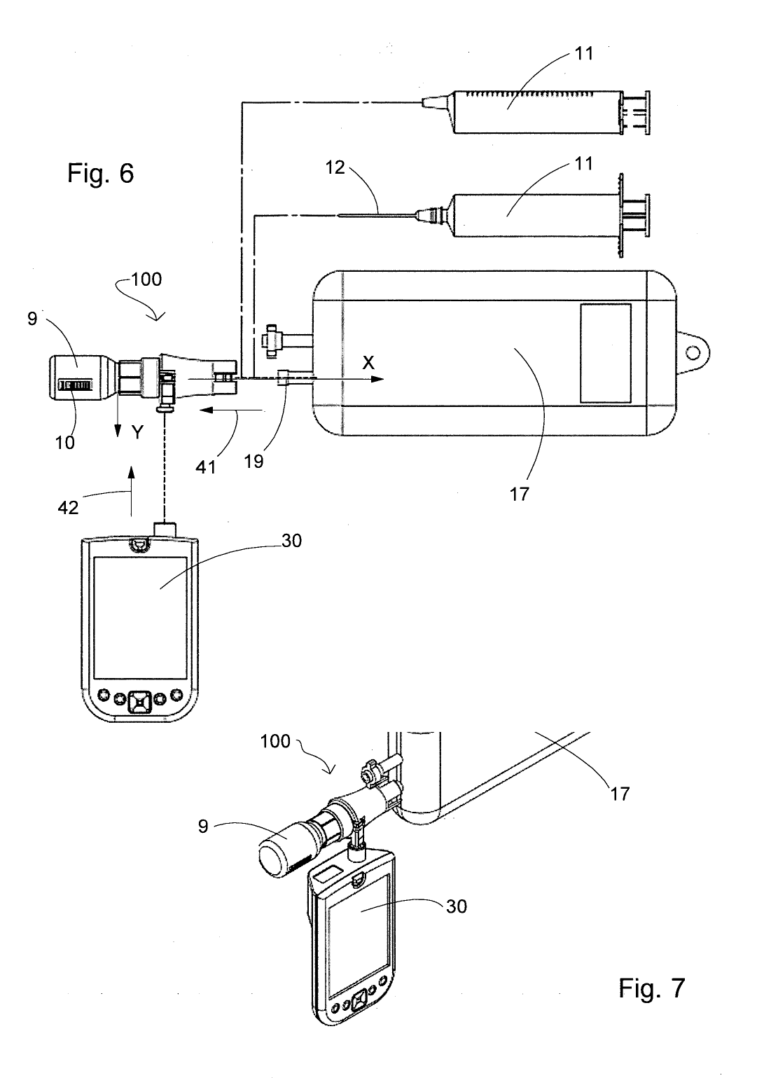

FIG. 6 is .a schematic side view of the exemplary embodiment of the drug port smart valve 100, according to the present invention, and of the HHD computer 30, an IV bag 17, a syringe 11, and a syringe 11 with a syringe needle 12.

This illustration shows the stages of connection of a drug mixing unit, such as an IV bag 17, a syringe 11, or a syringe 11 with a syringe needle 12, to the drug port smart valve 100. The connection is done by pushing in the direction of the arrow 41′, namely in the direction −x, of IV bag second port 19, in the case of connecting an IV bag 17, to the outlet connector, which is not shown in the present illustration. When this action is performed, drug vial 9 stays connected to the drug port smart valve 100.

FIG. 7 is a schematic perspective view of the exemplary embodiment of the drug port smart valve 100, according to the present invention, in a state in which the flow of fluid is blocked, and it is connected to a drug vial 9, an IV bag 17, and a HHD computer 30. The connection to the HHD computer 30, which is for the purpose of opening passage for fluid, is enabled after the vial barcode sticker 10 is scanned and its code is entered into the database.

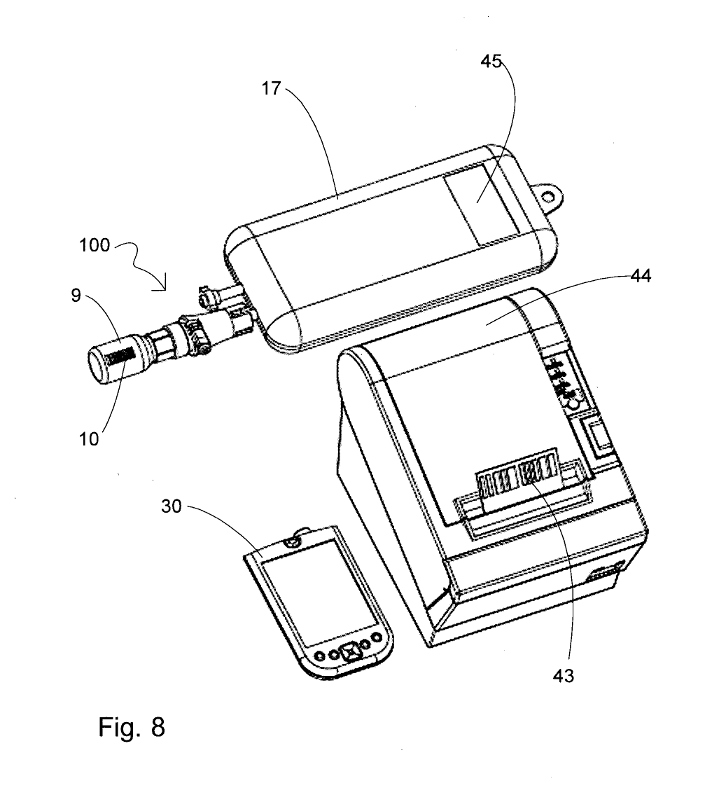

FIG. 8 is a schematic perspective view of the exemplary embodiment of the drug port smart valve 100, according to the present invention, which is connected to a drug vial 9, and an IV bag 17, alongside a HHD computer 30, and a barcode sticker printer 44. After opening passage for the flow of fluid, the HHD computer 30 is disconnected from the drug port smart valve 100, and is connected to the barcode sticker printer 44 by Bluetooth or IR radiation, or by any other suitable means, which produces barcode sticker 43, which bears a code identical to that of vial barcode sticker 10.

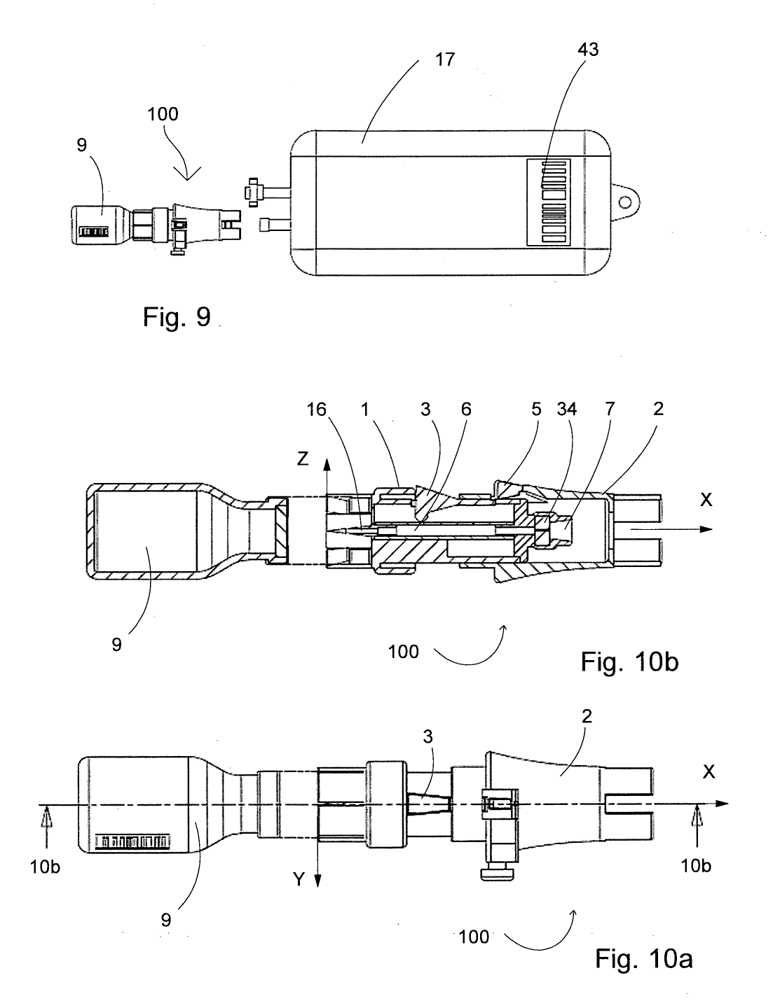

FIG. 9 is a schematic side view of the exemplary embodiment of the drug port smart valve 100, according to the present invention, which is connected to a drug vial 9, alongside an IV bag 17. At this stage, the barcode sticker 43 is stuck onto the IV bag 17.

FIG. 10a is a schematic side view of the exemplary embodiment of the drug port smart valve 100, according to the present invention, upon which section plane 10b-10b is marked, alongside a drug vial 9. This illustration once again shows the stages of blocking and opening the passage for fluid flow.

At first the drug vial 9 is driven towards the immoveable assembly 1, with shutter 3, and the moveable assembly 2, in rear position before motion.

As noted, shutter 3 is harnessed on its side, and is made of elastic material to enable its motion, which is mainly rotational movement of its free end, when force is applied to it, and also to enable its return to its original position should the applied force be removed.

FIG. 10b is a schematic cross sectional view 10b-10b of the first exemplary embodiment of the drug port smart valve 100, according to the present invention. The section shown in the illustration displays the following components: the immoveable assembly 1, the moveable assembly 2, the shutter 3, the breakable lock 5, the flexible tube 6, the outlet connector 7a , and a second seal 34, which can also be made of elastomer.

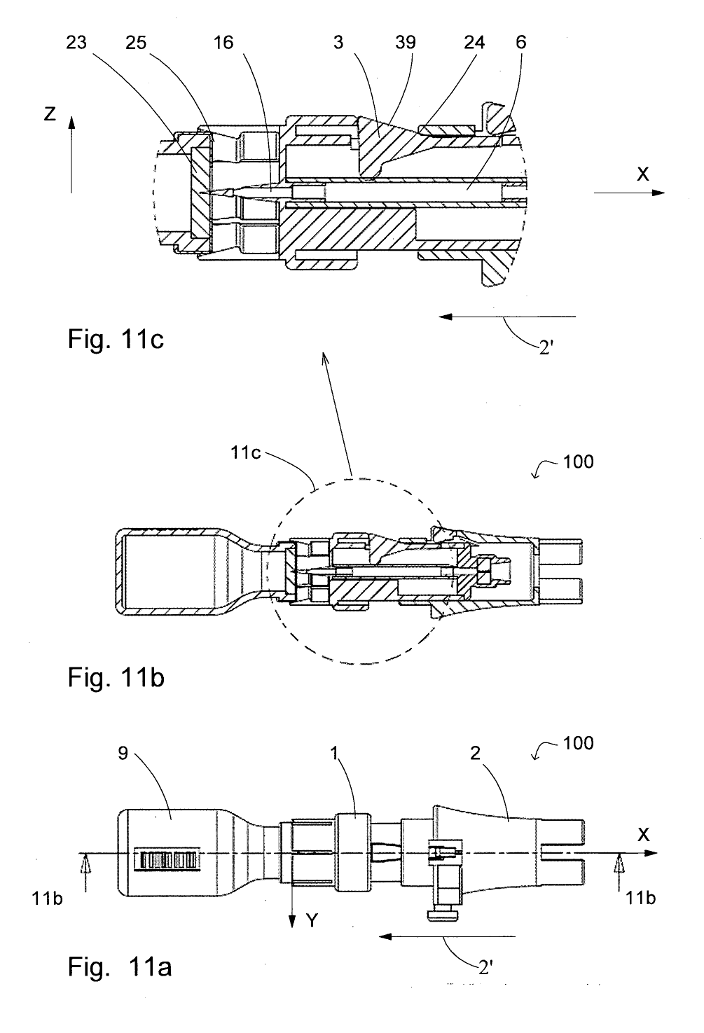

FIG. 11 a is a schematic side view of the exemplary embodiment of the drug port smart valve 100, according to the present invention, upon which section plane 11b-11b is marked. This illustration shows movement 2′ in the direction −x of the moveable assembly 2 as a result of the force applied to it.

FIG. 11b is a schematic cross sectional view 11b-11b of the exemplary embodiment of the drug port smart valve 100, according to the present invention. Circle 11c is marked on the illustration and the details of its contents will be magnified in FIG. 11c.

FIG. 11c is a schematic cross sectional view 11b-11b of the exemplary embodiment of the drug port smart valve 100, according to the present invention. The illustration shows a magnification of the contents of circle 11c. This illustration shows that movement 2′ in the direction −x of the moveable assembly 2 (as a result of force applied to it) applies force on shutter 3. As a result of the friction generated at point 24. Point 24 is a point moving along the shutter inclined plane 39, with the movement of the moveable assembly 2 with regard to the immoveable assembly 1. The movement of the end, shown in the left of the illustration, causes movement of the shutter 3 is in the direction −z towards the flexible tube 6, which blocks the tube. As a result of movement 2′, the first integral needle 16 is inserted into the internal volume of the drug vial 9 and is connected to the inlet port 25 through the drug vial seal 23. The puncturing of the drug vial seal 23 will enable flow of fluid into the flexible tube 6.

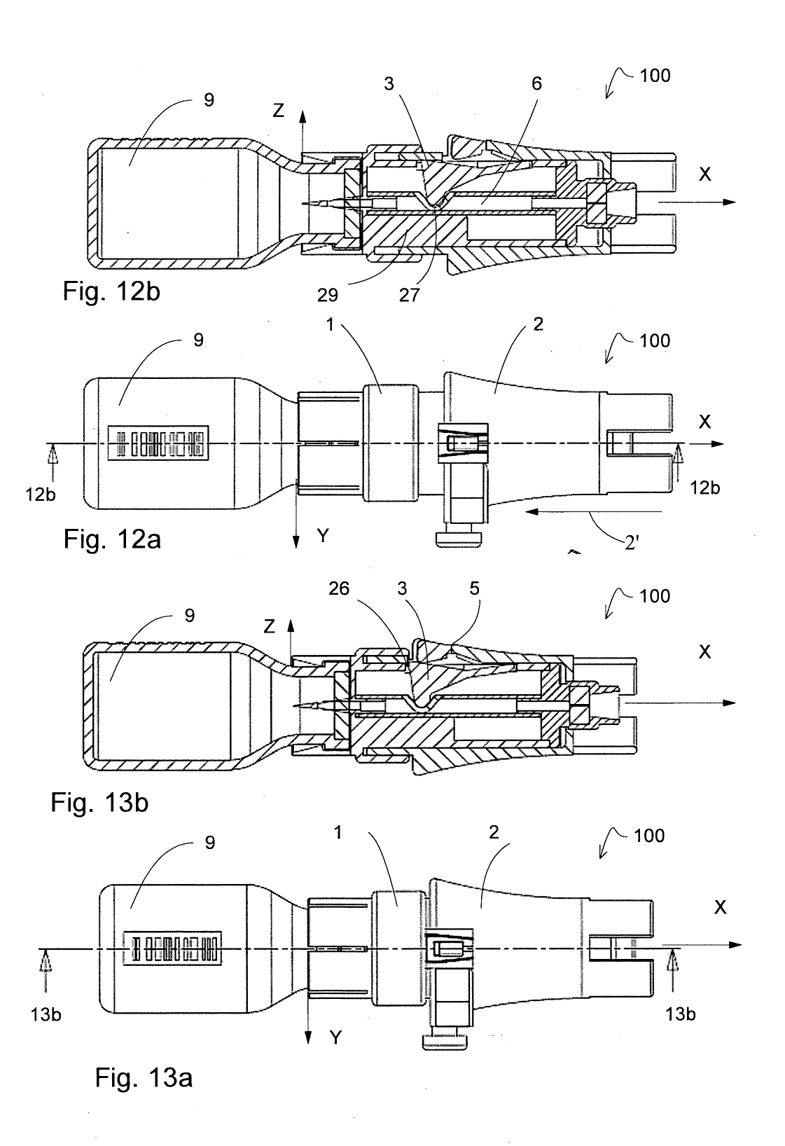

FIG. 12a is a schematic side view of the exemplary embodiment of the drug port smart valve 100, according to the present invention, upon which section plane 12b-12b is marked.

FIG. 12b is a schematic cross sectional view 12b-12b of the exemplary embodiment of the drug port smart valve 100, according to the present invention. The continued movement of the moveable assembly 2 in the direction −x with regard to the immoveable assembly 1 causes shutter 3 to press the flexible tube 6 towards the lower anvil 29, to create blockage of fluid flow through the flexible tube 6.

FIG. 13a is a schematic side view of the exemplary embodiment of the drug port smart valve 100, according to the present invention, upon which the section plane 13b-13b is marked.

FIG. 13b is a schematic cross sectional view 13b-13b of the exemplary embodiment of the drug port smart valve 100, according to the present invention. The movement of the moveable assembly 2 is obstructed by structural parts of the immoveable assembly 1 and the breakable lock 5 is unbroken and serves as a brake, locking the moveable assembly 2 and the shutter 3 which cannot move back, in the direction z.

Following the end of the motion described above, the drug port smart valve 100 will be impossible to open, namely it will be impossible to move the moveable assembly 2 in the direction x with regard to the immoveable assembly 1, seeing as shutter 3, which is a component mechanically connected to the immoveable assembly 1 is slightly released and by means of the shutter rear wall 26 blocks the moveable assembly 2 from moving in the direction x. Therefore, in this state the flexible tube 6 is blocked for the flow of fluid.

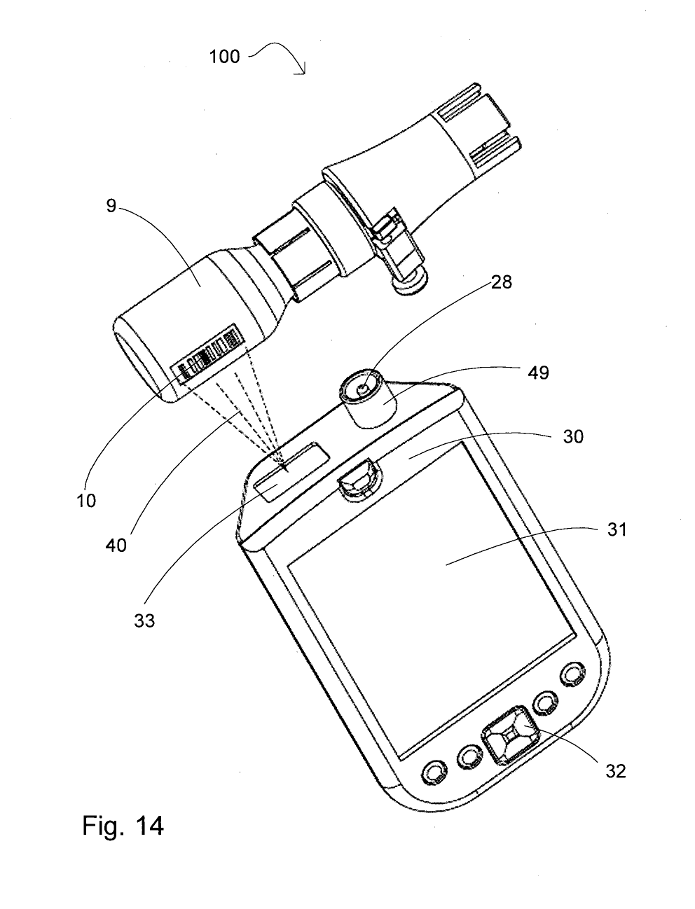

FIG. 14 is a schematic perspective view of the exemplary embodiment of the drug port smart valve 100, according to the present invention, and of a HHD computer 30. The illustration shows the stage of scanning vial barcode sticker 10 by the HHD computer 30. The scanning can also be by means of IR radiation 40, or other scanning devices. The illustration shows the following components of the HHD computer 30: the key pin 28, HHD computer port 49, LCD screen 31, and keypad 32.

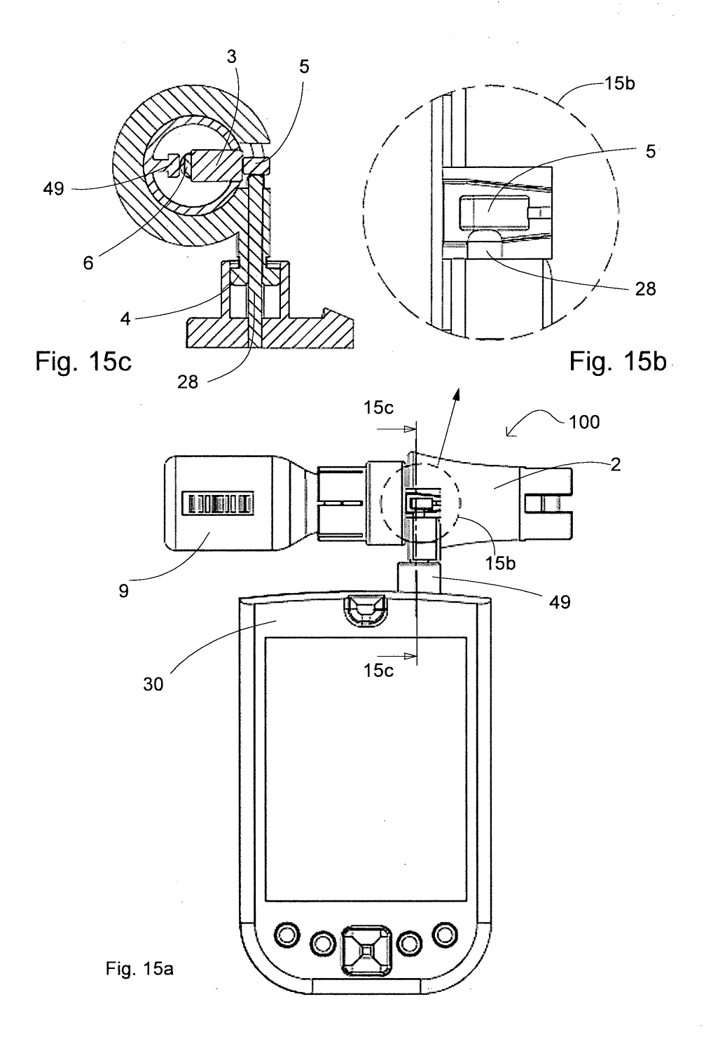

FIG. 15a is a schematic side view of the exemplary embodiment of the drug port smart valve 100, according to the present invention, upon which section plane 15c-15c is marked, and circle 12b which includes details that will be magnified in the next illustration.

The drug port smart valve 100 is connected to drug vial 9, and HHD computer 30.

FIG. 15b is a schematic side view of details of the exemplary embodiment of the drug port smart valve 100, according to the present invention, as shown in circle 12b. The illustration specifically shows key pin 28 touching the breakable lock 5.

FIG. 15c is a schematic cross sectional view 15c-15c of the exemplary embodiment of the drug port smart valve 100, according to the present invention.

Opening the passage for the flow of fluid through the flexible tube 6 is done by connecting the HHD computer 30 via the HHD computer port 49 into the opening port 4 of the moveable assembly 2. Thus, the key pin 28 can be activated in the direction of the breakable lock 5.

The flexible tube 6 at this stage is still closed, namely the blockage is maintained and fluid cannot flow through.

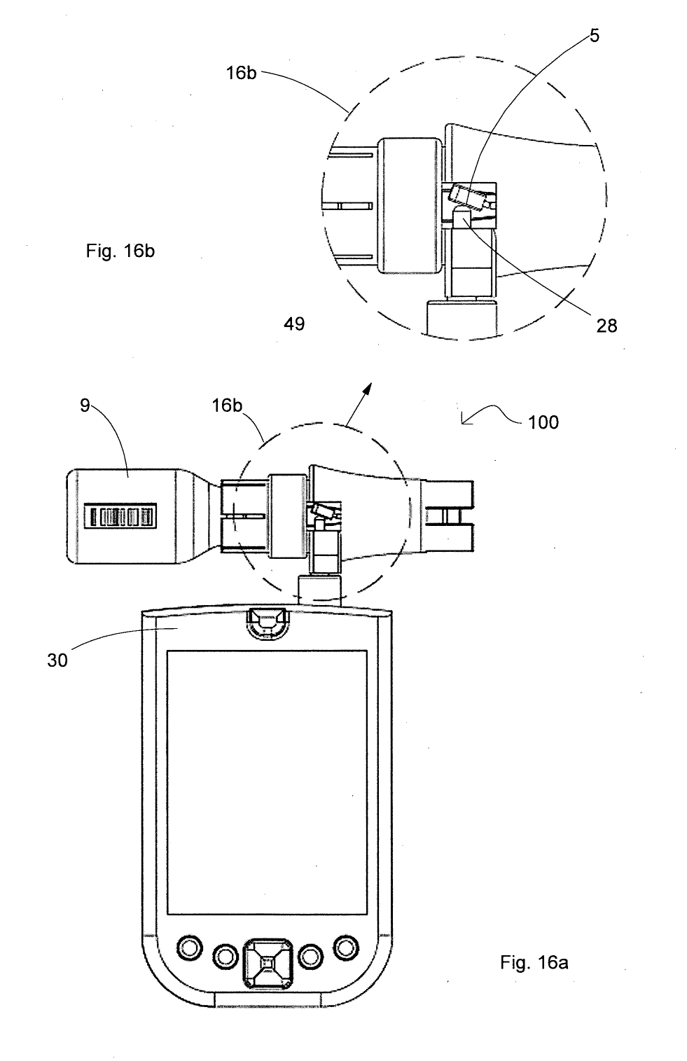

FIG. 16a is a schematic side view of the exemplary embodiment of the drug port smart valve 100, according to the present invention, marked with circle 16b, which includes details that will be magnified in the next illustration. The drug port smart valve 100 is connected to drug vial 9 and HHD computer 30.

FIG. 16b is a schematic side view of details of the exemplary embodiment of the drug port smart valve 100, according to the present invention, within circle 16b.

At this stage, the HHD computer 30 pushes the key pin 28 in the direction of the breakable lock 5, thus applying perpendicular force to the breakable lock 5, and breaking it.

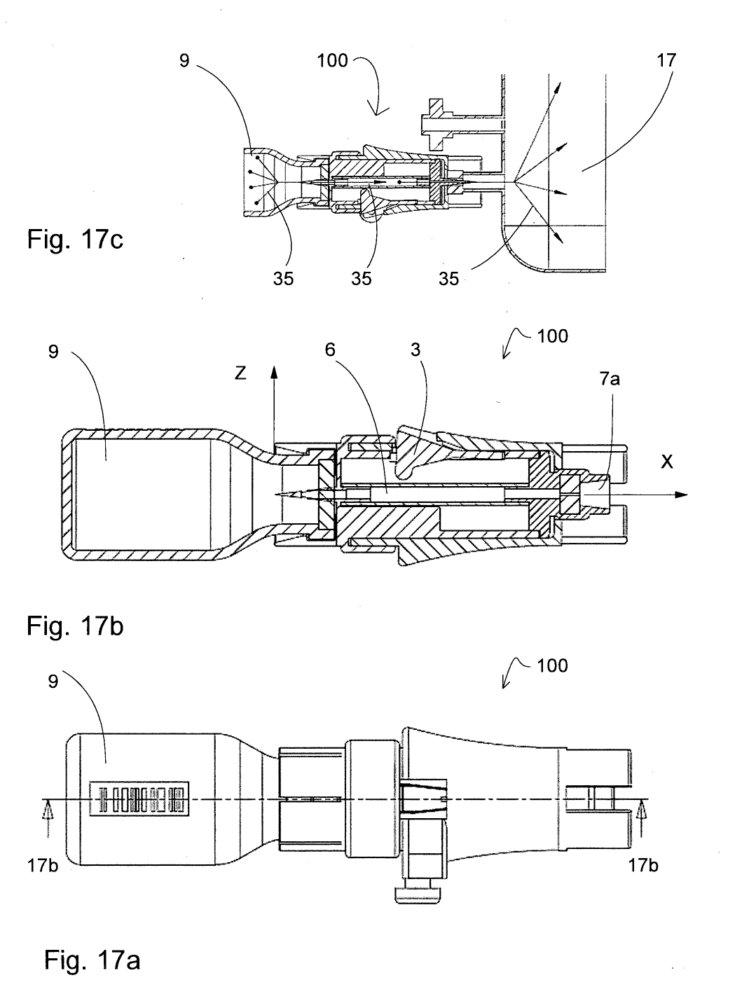

FIG. 17a is a schematic side view of the exemplary embodiment of the drug port smart valve 100, according to the present invention, on which section plane 17b-17b is marked, and which is connected to drug vial 9.

FIG. 17b is a schematic cross sectional view 17b-17b of the exemplary embodiment of the drug port smart valve 100, according to the present invention.

After breaking the breakable lock 5, there is nothing to prevent the shutter 3 from moving back to its original position. Indeed, its edge, shown in the left of the illustration, moves as a result of the force applied to it by the flexible tube 6, in addition to its elasticity which aspires to return it to its original state, without any bending forces. After the shutter 3 returns to its former state, the flexible tube 6 is opened for the flow of fluid so that liquid medication within the drug vial 9 can flow towards the outlet connector 7a.

FIG. 17c is a schematic cross sectional view 17b-17b of the first exemplary embodiment of the drug port smart valve 100, showing the flow of fluid 35 from drug vial 9, through the drug port smart valve 100, to IV bag 17.

The following three illustrations show three possible connections of the drug port smart valve, to various vessels serving as a source of medical fluids designated for injection into the body of a human or an animal, or any other suitable target. The first option is connection to an IV bag, the second is connection to a syringe without a needle, and the third is a connection to a syringe with a needle.

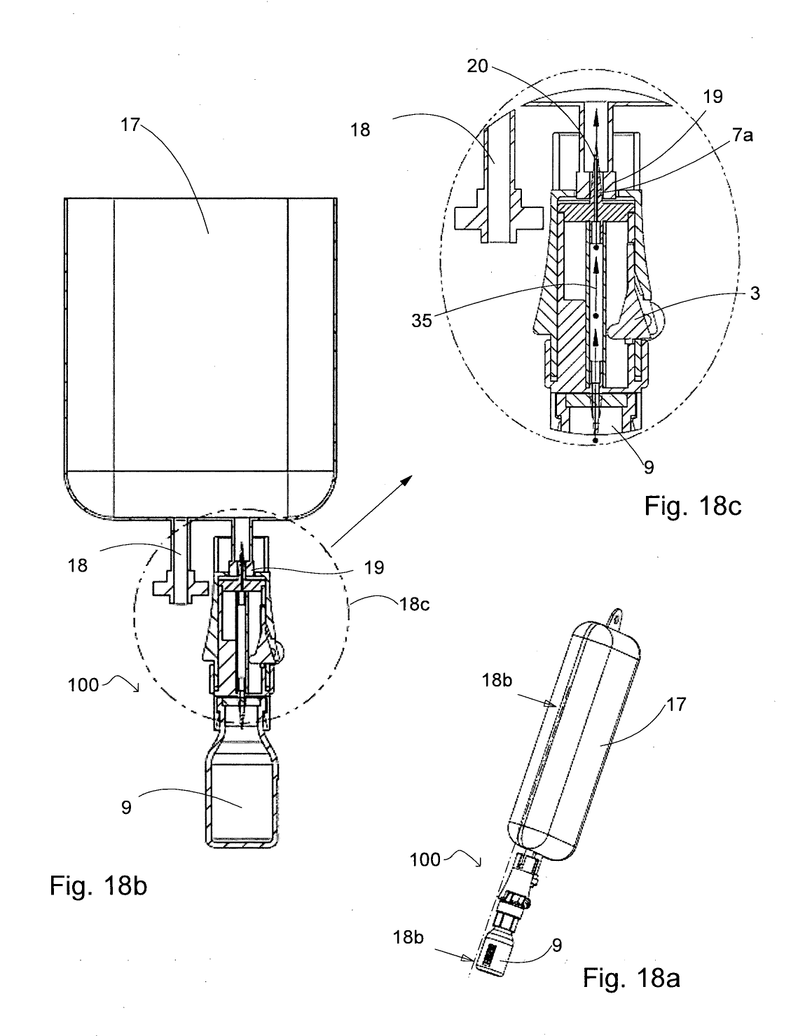

FIG. 18a is a schematic side view of the exemplary embodiment of the drug port smart valve 100, according to the present invention, which is connected to a drug vial 9 and an IV bag 17, on which section plane 18b-18b is marked.

FIG. 18b is a schematic cross sectional view 18b-18b of the exemplary embodiment of the drug port smart valve 100, according to the present invention, which is connected to a drug vial 9 and an IV bag 17, and which is marked with circle 18b, whose contents are magnified in FIG. 18c.

The illustration shows that the drug port smart valve 100 is connected to the IV bag 17 at IV bag second port 19, which serves as a designated entrance for injecting medication, and next to which IV bag first port 18 is disposed, serving as an exit for the passage of IV fluid, which could contain medication, to the tube which leads to the IV recipient.

FIG. 18c is a schematic cross sectional view of details of the exemplary embodiment of the drug port smart valve 100, according to the present invention, which is within circle 18c.

A second integral needle 20 disposed within immoveable assembly 1 is pushed with force to be inserted into the first port 18. The insertion punctures a diaphragm, designated for this purpose, in the IV bag. After connection, fluid can go through first port 18, namely there is fluid free flow 35, from the drug vial 9 through the drug port smart valve 100 to the IV bag 17.

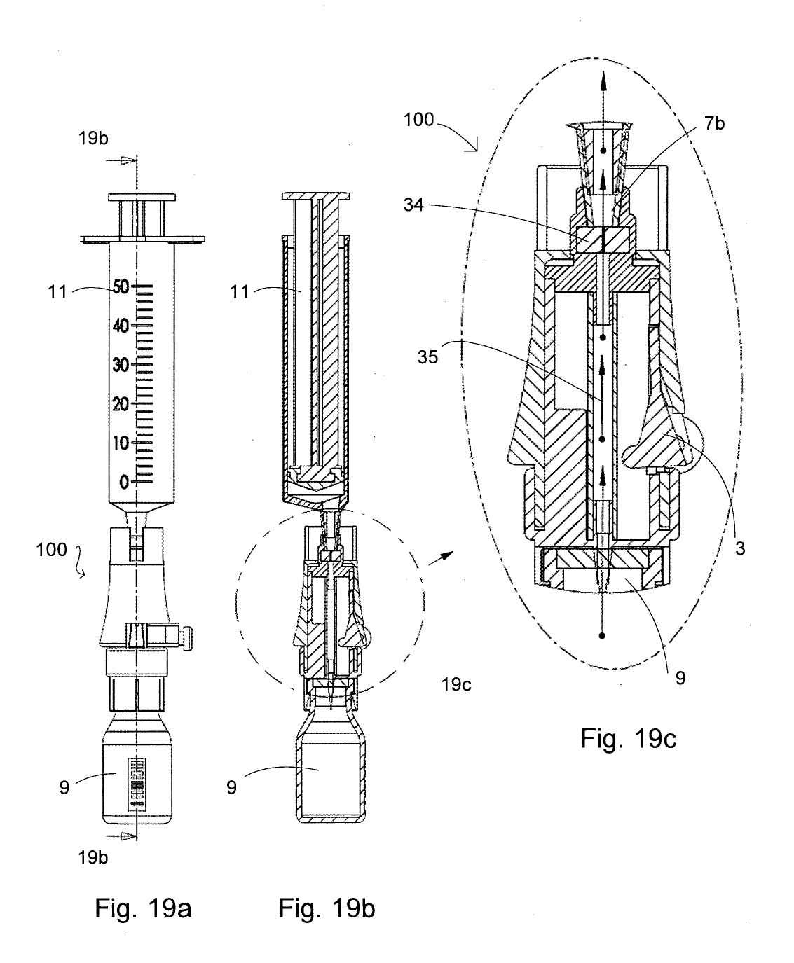

FIG. 19a is a schematic side view of a second exemplary embodiment of the drug port smart valve 100, according to the present invention, which is connected to a drug vial 9 and a syringe 11 without a needle, and which is marked with section plane 19b-19b.

FIG. 19b is a schematic cross sectional view 19b-19b of the second exemplary embodiment of the drug port smart valve 100, according to the present invention, which is connected to a drug vial 9 and a syringe 11 without a needle, and which is marked with circle 19c, whose contents are magnified in FIG. 19c.

FIG. 19c is a schematic cross sectional view of details of the second exemplary embodiment of the drug port smart valve 100, according to the present invention, which are within circle 19c.

During connection, the syringe 11 without the needle is inserted into the outlet connector 7b , in which the second seal 34, which can also be made of elastomer, is disposed. When the syringe 11 without the needle is pushed with sufficient force, it penetrates the second seal 34 and the syringe 11 can be activated to suck fluid from the drug vial 9. This fluid can now travel by free flow 35 through the drug port smart valve 100.

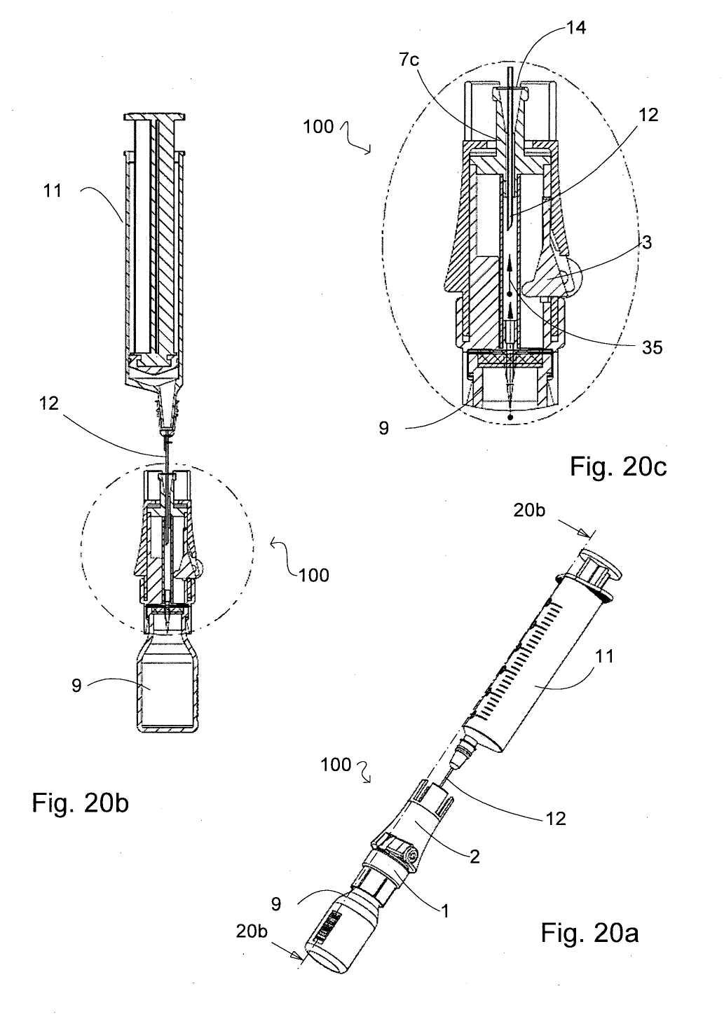

FIG. 20a is a schematic perspective view of a third exemplary embodiment of the drug port smart valve 100, according to the present invention, connected to a drug vial 9 and to a syringe 11 equipped with a syringe needle 12. The illustration shows section plane 20b-20b.

FIG. 20b is a schematic cross sectional view 20b-20b of the third exemplary embodiment of the drug port smart valve 100, according to the present invention, which is connected to a drug vial 9 and a syringe 11 equipped with a syringe needle 12, and which is marked with circle 20c, whose contents are magnified in FIG. 20c.

FIG. 20c is a schematic cross sectional view of details of the third exemplary embodiment of the drug port smart valve 100, according to the present invention, within circle 20c. As shown in the illustration, the syringe needle 12 is inserted through a diaphragm 14 disposed at the end of the outlet connector 7c , enabling the suction of fluid by means of the syringe 11, with fluid free flow 35 enabled during the suction process.

The following three pairs of illustrations are for comparative views of the first, second, and third exemplary embodiment, according to the present invention, to enable observing the differences between them.

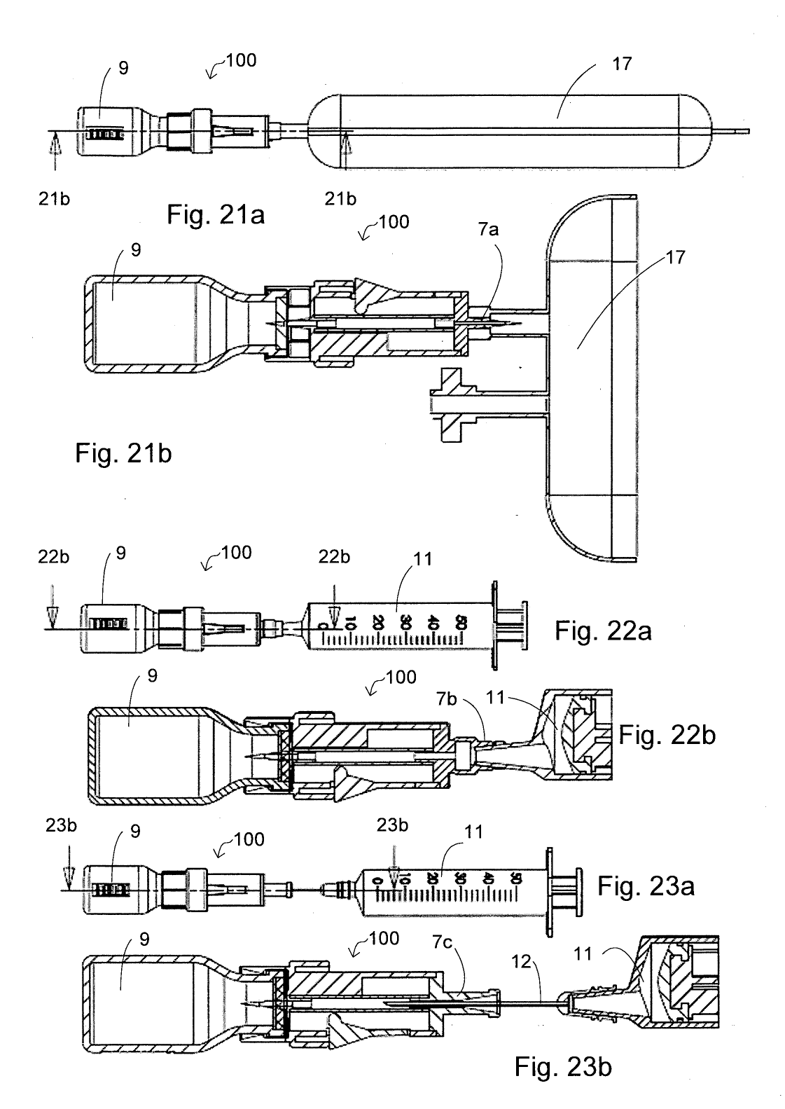

FIG. 21 a is schematic side view of the first exemplary embodiment of the drug port smart valve 100, according to the present invention, connected to a drug vial 9 and to an IV bag 17. The illustration shows section plane 21b-21b.

FIG. 21b is a schematic cross sectional view 21b-21b of the first exemplary embodiment of the drug port smart valve 100, according to the present invention, connected to a drug vial 9 and to an IV bag 17. The illustration shows outlet connector 7a of the first exemplary embodiment of the drug port smart valve 100.

FIG. 22a is schematic side view of the second exemplary embodiment of the drug port smart valve 100, according to the present invention, connected to a drug vial 9 and to a syringe 11 without a needle. The illustration shows section plane 22b-22b.

FIG. 22b is a schematic cross sectional view 22b-22b of the second exemplary embodiment of the drug port smart valve 100, according to the present invention, connected to a drug vial 9 and to a syringe 11 without a needle. The illustration shows outlet connector 7b of the second exemplary embodiment of the drug port smart valve 100.

FIG. 23a is schematic side view of the second exemplary embodiment of the drug port smart valve 100, according to the present invention, connected to a drug vial 9 and to a syringe 11 equipped with a syringe needle 12. The illustration shows section plane 23b-23b.

FIG. 23b is a schematic cross sectional view 23b-23b of the second exemplary embodiment of the drug port smart valve 100, according to the present invention, connected to a drug vial 9 and to a syringe 11 equipped with a syringe needle 12. The illustration shows outlet connector 7c of the third exemplary embodiment of the drug port smart valve 100.

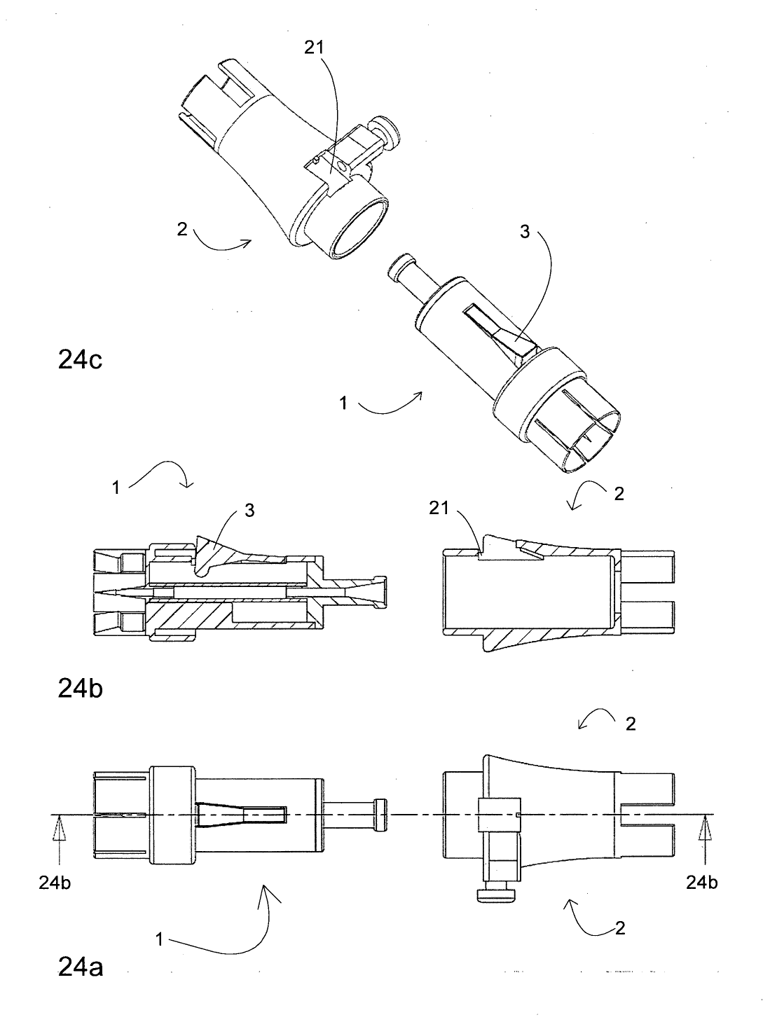

FIG. 24a is a schematic side view of both assemblies of the exemplary embodiment of the drug port smart valve, the immoveable assembly 1, and the moveable assembly 2, according to the present invention, when they are separated from each other, on which section plane 24b-24b is marked.

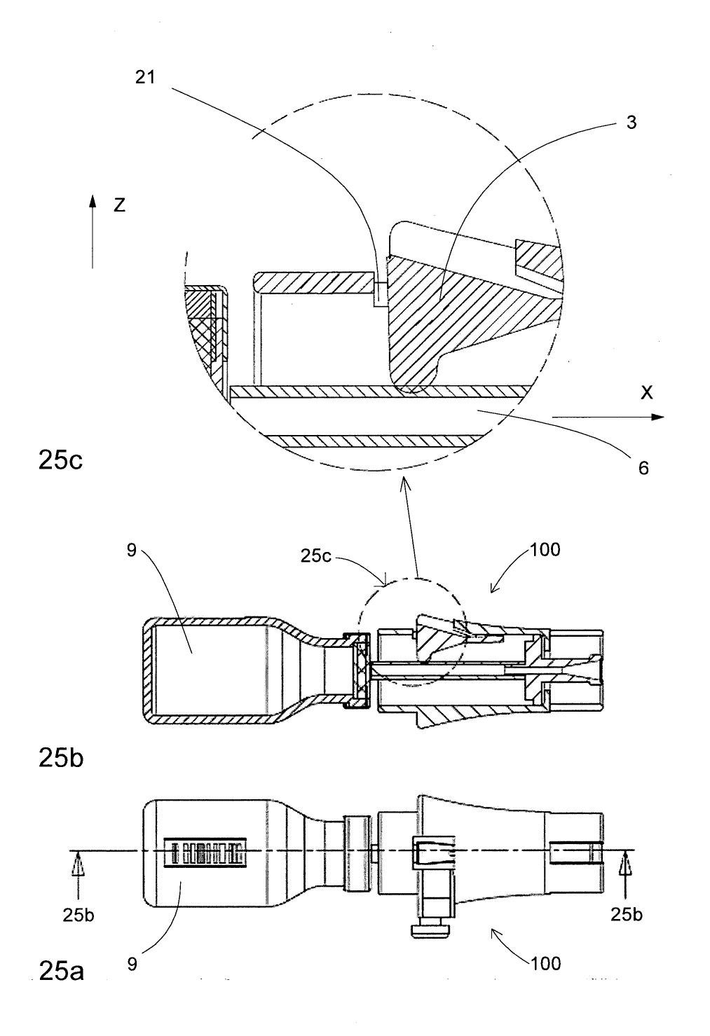

FIG. 24b is schematic cross sectional view 24b-24b of both assemblies of the exemplary embodiment of the drug port smart valve, the immoveable assembly 1, and the moveable assembly 2, according to the present invention, when they are separated from each other. This illustration clearly shows the various components and their orientation to both assemblies. The illustration particularly indicates the shutter 3, and window 21, whose designation will be described in the specification of FIG. 25c.

FIG. 24c is a schematic perspective view of both assemblies of the exemplary embodiment of the drug port smart valve, the immoveable assembly 1, and the moveable assembly 2, according to the present invention, when they are separated from each other. The illustration particularly indicates the shutter 3, and the window 21.

FIG. 25a is a schematic side view of the exemplary embodiment of the drug port smart valve 100, according to the present invention, on which section plane 24b-24 is marked, and which is connected to drug vial 9.

FIG. 25b is a schematic cross sectional view 25b-25b of the exemplary embodiment of the drug port smart valve 100, according to the present invention and which is connected to drug vial 9. Circle 25c is marked on the illustration and the details of its contents will be magnified in FIG. 25c.

FIG. 25c is a schematic cross sectional view 25b-25b of the exemplary embodiment of the drug port smart valve 100, according to the present invention. The illustration shows a magnification of the contents of circle 25c. This illustration shows how both assemblies lock to each other. When window 21 passes shutter 3, moving in direction −x, it is released and then moves upwards, in direction z, as a result of the force applied by the flexible tube 6. After the highest point of shutter 3 passes the corner of window 21, a lock is achieved, and the moveable assembly 2 cannot move back in direction x.

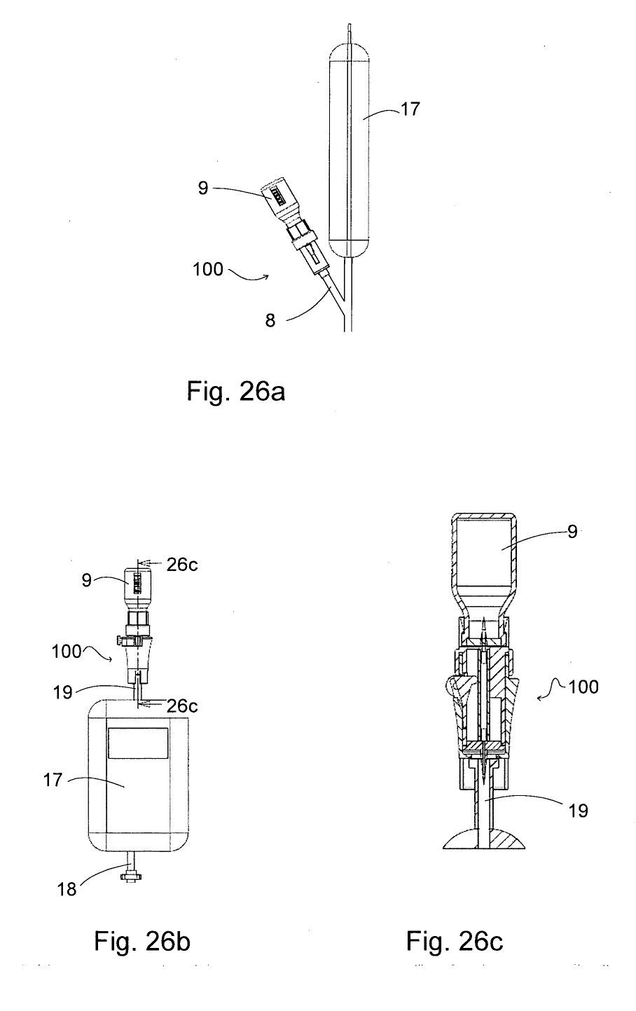

FIG. 26a is a schematic side view of an exemplary embodiment of the drug port smart valve 100, according to the present invention, connected to a drug vial 9 and to an “on-line” port 8. This illustration demonstrates the wide variety of options for connection of the drug port smart valve 100 to various means serving for administration of medication.

FIG. 26b is a schematic side view of an exemplary embodiment of the drug port smart valve 100, according to the present invention, connected to a drug vial 9 and to an IV bag 17. This illustration demonstrates additional options for connection of the drug port smart valve 100 to IV bag 17. In this case, the connection is to IV bag second port 19 on the opposite side of IV bag first port 18. The illustration shows section plane 26c-26c.

FIG. 26c is a schematic cross sectional view of an exemplary embodiment of the drug port smart valve 100, according to the present invention, connected to a drug vial 9 and to an IV bag 17.

While the invention has been described with respect to a limited number of embodiments, it will be appreciated that many variations, modifications and other applications of the invention may be made, such as the verification of movement of fluids in all directions from bags to vials, bags to syringes, and syringes to vials.

What is claimed is:

1. A drug port valve for ensuring authentication of medications, in fluid state, infused in IV bags and syringes from a drug vial, under control of an authentication unit, the authentication unit containing characteristics of the medication fluid and details of the patient, for calculating a correlation value between the details and the characteristics and having a key for opening a pass for the medication fluid flow through the drug port valve, the drug port valve comprising:
(a) an immoveable assembly including:
(i) a breakable lock;
(ii) a flexible tube having first end and second end, wherein said first end of said flexible tube is disposed on said immoveable assembly; and
(iii) an outlet connector disposed on said second end of said flexible tube,
wherein said breakable lock can be broken by a key pin, locking said immoveable assembly; and
(b) a moveable assembly having limited linear, one dimensional, motion capability, with relation to said immoveable assembly, said immoveable assembly including:
a shutter.

2. The drug port valve of claim 1, wherein the immoveable assembly further includes:
(ii) an inlet adaptor having external end, shape, and dimensions enabling the connection of a drug vial.

3. The drug port valve of claim 2, wherein the moveable assembly further includes:
(ii) a first integral needle having a sharp tip for enabling puncturing said drug vial's seal for enabling flow of fluid from said drug vial into said flexible tube, as a result of movement of said moveable assembly, wherein said immoveable assembly defines a three-dimensional XYZ Cartesian coordinate system, having an origin located at a geometrical center of said external end of said inlet adaptor, wherein an X axis is directed from said origin into said drug port valve, and goes through said sharp tip of said first integral needle, wherein a Z axis is perpendicular to said X axis and is on a symmetry plane with said shutter, and wherein a Y axis is perpendicular to both prior axes.

4. The drug port valve of claim 3, wherein said drug port valve is configured so as to cause said shutter to press said flexible tube and to create blockage of fluid flow through said flexible tube as a result of movement of said moveable assembly in the direction −x with regard to said immoveable assembly.

5. The drug port valve of claim 4, wherein said drug port valve is further configured such that breaking of said breakable lock causes said shutter to cease pressing said flexible tube and to open said flexible tube for flow of fluid.

6. The drug port valve of claim 5, wherein said outlet connector is configured so as to enable connection to an IV bag.

7. The drug port valve of claim 5, wherein said outlet connector is configured so as to enable connection to a syringe without a needle.

8. The drug port valve of claim 5, wherein said outlet connector is configured so as to enable connection to a syringe with a needle.

9. A method for ensuring authentication of medications, in fluid state, infused in IV bags a drug vial the method comprising the stages of:
(a) providing a drug port valve, said drug port valve including:
(i) an immoveable assembly including:
(A) a breakable lock;
(B) a flexible tube having first end and second end, wherein said first end of said flexible tube is disposed on said immoveable assembly; and
(C) an outlet connector disposed on said second end of said flexible tube, wherein said breakable lock can be broken by a key pin, locking said immoveable assembly; and
(ii) a moveable assembly having limited linear, one dimensional, motion capability, with relation to said immoveable assembly, said immoveable assembly including:
(A) a shutter;
(b) connecting a drug vial, having a vial barcode sticker having a drug vial barcode sticker code, to said drug port valve;
(c) pressing on and closing said flexible tube;
(d) scanning said vial barcode sticker by an HHD computer;
(e) connecting a IV bag to said drug port valve;
(f) using said HHD computer for releasing the pressing and opening said flexible tube to fluid flow; and
(g) printing a second barcode sticker with a code identical to said drug vial barcode sticker code, and sticking said second barcode sticker on said IV bag.
